(19) United States
(12) Patent Application Publication
(10) Pub. No.: US 2009/0205669 A1
(43) Pub. Date: Aug. 20, 2009

(54) APPARATUS FOR INFECTION CONTROL OF A SHOULDER POSITION DEVICE

(76) Inventors: Michael Campagna, Oak Park, IL (US); Jonathan S. Citow, Glencoe, IL (US)

Correspondence Address: MICHAEL BEST & FRIEDRICH LLP, Two Prudential Plaza, 180 North Stetson Avenue, Suite 2000, CHICAGO, IL, 60601, US

(21) Appl. No.: 12/031,373

(22) Filed: Feb. 14, 2008

(51) Int. Cl. A61B 19/02

(52) U.S. Cl. 128/849; 150/154

(57) ABSTRACT

A disposable sleeve adapted to be applied to a shoulder position device. The disposable sleeve includes a first flexible side wall, a second flexible side wall connected to the first flexible side wall at a first edge and a second edge, and an opening at a first end of the sleeve, the opening adapted to receive the shoulder position device. The disposable sleeve also includes a pad secured to a second end of the sleeve, the pad defining a radius of curvature, and a plurality of tabs secured to the sleeve, the tabs having a layer of adhesive adapted to be secured to a portion of the shoulder position device.

### BACKGROUND

Staphylococcus aureus or Staph aureus is a bacteria found on everyone's skin. It is also found most anywhere in the environment. Most of the time, this bacteria lives on the skin without causing any problems. However, this bacteria may creep under the skin through a bug bite or scratch and cause an infection. It also may be inhaled and cause pneumonia although this is rather uncommon in children and adults.

Most Staph are sensitive to simple antibiotics. Unfortunately, the overuse of antibiotics has made these simple Staph resistant to some antibiotics and thus, a little harder to kill. In some hospitals (particularly intensive care units) and nursing homes where antibiotic use is often high, there is a higher prevalence of resistant Staph. When Staph become resistant to methicillin (a simple type of penicillin), they are given the name MRSA, which stands for methicillin-resistant Staphylococcus aureus.

Most healthy people have strong immune systems and are able to fight off a Staphylococcus aureus infection themselves and have only mild symptoms. However, people with weakened immune systems (due to other illnesses) or who have undergone surgery (for example heart surgery or hip replacement) can develop more serious problems. In addition, people who are hospitalized for long periods or are seriously ill in intensive care are at risk for a staph infection. In more vulnerable people, Staphylococcus aureus bacteria have been known to cause boils, abscesses, impetigo, septic wounds, heart-valve problems and toxic shock syndrome. In extreme cases, it can result in death.

Because staphylococcus aureus bacteria live on the skin it is easily spread by direct skin contact, often on people's hands. MRSA is also spread on bedding, towels, clothing and equipment. This is one reason why strict hygiene measures in hospitals are so important.

### SUMMARY

MRSA is mostly an infection that people get in hospital. As reported in the news the number of reported MRSA infections has increased in recent years. The reason for the rise is due to many things, such as new strains of MRSA being more powerful, an increased number of very sick people in the hospital, healthcare treatment being more complex—the use of central lines and catheters are much more widespread, patients move within and between hospitals more often, and high workloads which can make staff less able to stick to regular hand washing routines.

Hospital staff work to control the spread of MRSA by making sure they wash their hands and use cleansing alcohol hand gel between patients, screening patients for MRSA when or before they are admitted to the hospital, using antibiotics carefully in line with approved guidelines, improving cleaning and inspection of wards, looking after people with MRSA in single rooms until their infection has cleared, and having a policy on how to manage MRSA.

Additional measures can be utilized by the proper sterilization of medical or other devices used in hospitals and doctors' offices. Further, medical or other devices can be covered or protected with disposable covers or drapes to prevent the transfer of MRSA.

The present invention relates generally to the field of disposable sleeves applied to devices to prevent the transfer of bacteria in both clinical and intraoperative settings. The disposable sleeve is utilized to prevent the possibility of patient to patient pathogen migration thru usage of a medical or other device, such as a shoulder press utilized during X-ray imaging of a patient. The disposable sleeve is a radiolucent barrier sleeve or sheath, equipped with soft foam cushions to eliminate skin abrasion. The disposable sleeve includes pull-tabs that expose an adhesive layer to secure the sheath to the medical or other device outside of the x-ray field, allowing for manipulation without slippage. The disposable sleeve can be provided in a sterile or a non-sterile condition.

Other aspects of the invention will become apparent by consideration of the detailed description and accompanying drawings.

### BRIEF DESCRIPTION OF THE DRAWINGS

FIG. 1 is a side view of a human spine.

FIG. 2 is top view of a position device.

FIG. 3 is a rear view of the position device illustrated in FIG. 2.

FIG. 4 is a side view of an arm of the position device illustrated in FIG. 2.

FIG. 5 is an enlarged side view of the arm illustrated in FIG. 4.

FIG. 6 is a rear view of the arm illustrated in FIG. 4

FIG. 7 is a perspective view of the position device.

FIG. 8 is a perspective view of the position device including a disposable sleeve.

FIG. 9 is a perspective view of a disposable sleeve according to one embodiment of the present invention.

FIG. 10 is a top view of the disposable sleeve illustrated in FIG. 8.

FIG. 11 is a top view of the disposable sleeve illustrated in FIG. 8.

FIG. 12 is a top view of the disposable sleeve illustrated in FIG. 8.

FIG. 13 is a perspective view of the disposable sleeve applied to the position device.

FIG. 14 is an enlarged perspective view of the disposable sleeve applied to the position device.

FIG. 15 is a perspective view of a disposable sleeve for use with a position device.

FIG. 16 is a front view of a disposable sleeve for use with a position device.

FIG. 17 is a front view of a disposable sleeve for use with a position device.

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting.

Although directional references, such as upper, lower, downward, upward, rearward, bottom, front, rear, etc., may be made herein in describing the drawings, these references are made relative to the drawings (as normally viewed) for convenience. These directions are not intended to be taken literally or limit the present invention in any form. In addition, terms such as “first,” “second,” and “third” are used herein for purposes of description and are not intended to indicate or imply relative importance or significance.

The human spine or backbone is known medically as the vertebral column. Its role is to support the whole body, be capable of bending and twisting in all directions, and at the same time protect the vital structures such as nerves that run through it.

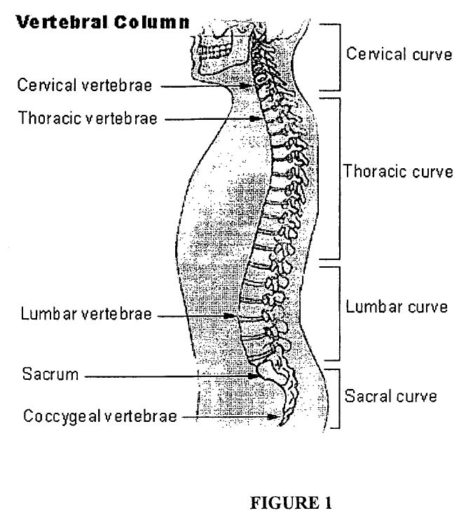

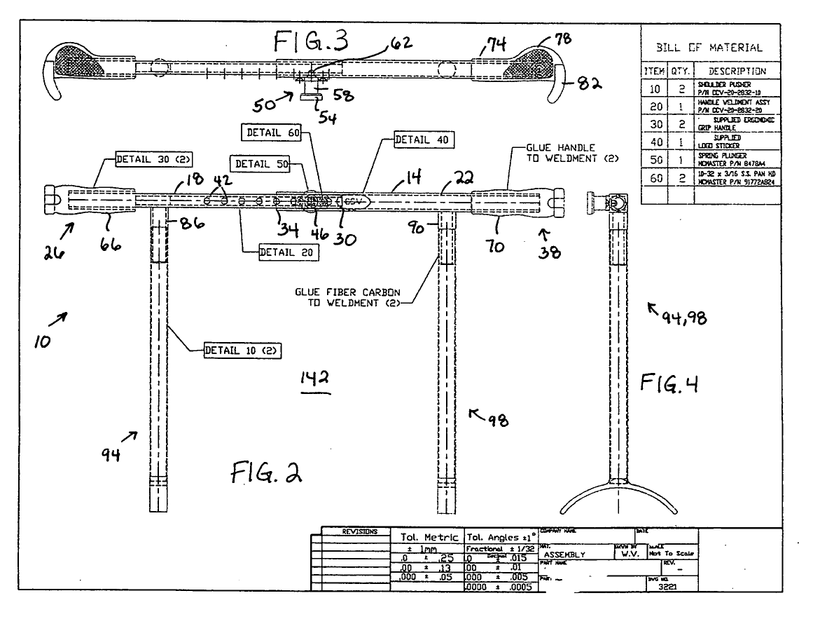

As illustrated in FIG. 1, the human spine consists of a column of bony blocks known as vertebrae, which sit one on top of another, joined together by tough ligaments to form the vertebral column. The spine is divided into several sections known as the cervical vertebrae, the thoracic vertebrae, the lumbar vertebrae, the sacrum, and the coccygeal vertebrae.

A device has been developed that optimizes intraoperative radiography by removing obstructions in the radiographic area of the cervical vertebrae. The device holds the subject's shoulders in a position so that all of the cervical vertebrae are visible in an X-ray.

The device is a shoulder press for use during the acquisition of X-rays of the cervical or cervical thoracic junction regions of the patient. The device optimizes the radiographic area of the patient during acquisition of X-rays. The device supports the patient's shoulders and adjusts the position of the shoulders such that they do not obstruct the X-ray images. The device is suitable for use in clinical and ER settings as well as for intraoperative use such as anterior cervical discectomy or fusion procedures.

The cervical vertebrae include seven vertebra and eight pairs of cervical nerves. The cervical vertebrae are those vertebrae in the neck, immediately behind the skull. The identified as C1 through C8. The thoracic vertebrae include twelve vertebra, identified as T1 through T12, in the upper and middle back. The lumbar vertebrae include five vertebra, identified as L1 through L5, in the lower part of the spine. The fifth lumbar vertebra sits on the sacrum, which in turn is connected to the coccyx, i.e., the tail bone. The sacrum consists of several vertebrae that have joined together. The sacrum is joined at its edges to the pelvis; the ring of bone that carries the trunk and which in turn is supported by the hips.

During anterior cervical disectomy and fusion procedures, X-rays may be necessary. The radiographic area in such a surgical procedure is constrained by anatomical obstruction by the subject's shoulders or the subject's jaw. It is rare that all seven cervical vertebrae are visible in a single X-ray because the jaw of the subject may obstruct the view of C1 and C2, while the shoulders of the subject may obscure C7 and the first thoracic vertebra T1.

In intraoperative and trauma situations related to the neck area of the spine, a physician needs to review anterior and lateral X-ray views of the cervical vertebrae. However, the cervical vertebrae are often obscured by the anatomical position of the patient's shoulders and/or jaw. It is rare that all seven cervical vertebra are visible in a single X-ray of the cervical vertebrae and/or cervical-thoracic junction. Typically, the patient's jaw obstructs the cervical vertebrae C1 and C2, and the patient's shoulders obstruct the cervical vertebra C7 and/or the thoracic vertebra T1.

FIG. 2 illustrates a position device 10 adapted to push the patient's shoulders caudally to allow a more inclusive view of the cervical vertebrae in anterior and lateral cervical spine films. The position device 10 or at least a portion of the device 10 is comprised of a radiolucent material, such as carbon fiber. Other suitable radiolucent materials also can be utilized in the device 10. The radiolucent material prevents the device 10 or portions of the device 10 from appearing in the X-ray film, i.e., the device 10 does not obstruct the radiographic field of view of the image. The position device 10 can provide correct level confirmation following interbody device placement, proper plate placement, and for post procedure confirmation.

The position device 10 includes an elongated rod 14 having a first portion 18 and a second portion 22. The first portion 18 includes a first end 26 and a second end 30 and has a first diameter. The second portion 22 includes a first end 34 and a second end 38 and has a second diameter. The first diameter and the second diameter are different such that the second end 30 of the first portion 18 slides within the first end 34 of the second portion 22. In other words, the elongated rod 14 telescopes to adjust to different lengths. The first portion 18 or the second portion 22 can include bearings to assist the telescoping action. In another construction, the interiors of the first portion 18 and the second portion 22 can include a powder coat to assist the telescoping action. In some constructions, the exteriors of the first portion 18 and the second portion 22 can include a coating, such as chrome or other suitable specialty coating. The coating can assist in the cleaning and/or sterilization process of the device 10.

The first portion 18 includes a plurality of apertures 42 positioned along its length. The second portion 22 includes an aperture 46 positioned near the first end 34. The first portion 18 and the second portion 22 of the elongated rod 14 have a substantially square cross-section, but other shapes for the cross-section are also possible.

The position device 10 also includes a knob 50 coupled to the second portion 22 of the elongated rod 14. The knob 50 includes a base 54, a plunger 58, and a protrusion 62 extending from the plunger 58. The plunger 58 can include a spring 62 or other elastic component. The protrusion 62 is sized to be received within the aperture 46 of the second portion 22 and the apertures 42 of the first portion 18. The knob 50 is manipulated to adjust the length of the elongated rod 14. More specifically, the knob 50 is manipulated to pull the plunger 58 to remove the protrusion 62 from the apertures 42, 46 such that the first portion 18 can telescope within the second portion 22. The knob 50 is then released to position the protrusion 62 into the desired apertures 42, 46 (i.e., the apertures 46 is aligned with one of the apertures 42).

The position device 10 also includes a first handle 66 mounted to the first end 26 of the first portion 18 of the elongated rod 14 and a second handle 70 mounted to the second end 38 of the second portion 22 of the elongated rod 14. The first handle 66 and the second handle 70 include ergonomic grips with lateral stability for proper placement of the device 10. The first handle 66 and the second handle 70 include an elongated portion 74 and an expanded portion 78 extending from and integral to the elongated portion 74. The first handle 66 and the second handle 70 also include an extension 82 extending from and integral to the expanded portion 78. The extension 82 narrows in cross-section as it extends further from the expanded portion 78. The extension 82 curves downward and toward the opposite end of the elongated rod 14. The first handle 66 and the second handle 70 can comprise rubber, silicone, or any other suitable material and/or combination of materials.

The position device 10 includes a first coupling 86 connected to and extending from the first portion 18 of the elongated rod 14 and a second coupling 90 connected to and extending from the second portion 22 of the elongated rod 14. The first coupling 86 and the second coupling 90 are oriented substantially perpendicular with respect to the elongated rod 14. The position device 10 also includes a first arm 94 and a second arm 98 removable coupled to the first coupling 86 and the second coupling 90, respectively. The first arm 94 and the second arm 98 substantially include the same structure, therefore only the first arm 94 is described herein. In another construction, the first arm 94 can be directly connected to and extend from the first portion 18 of the elongated rod 14 and, the second arm 98 can be directly connected to and extend from the second portion 22 of the elongated rod 14.

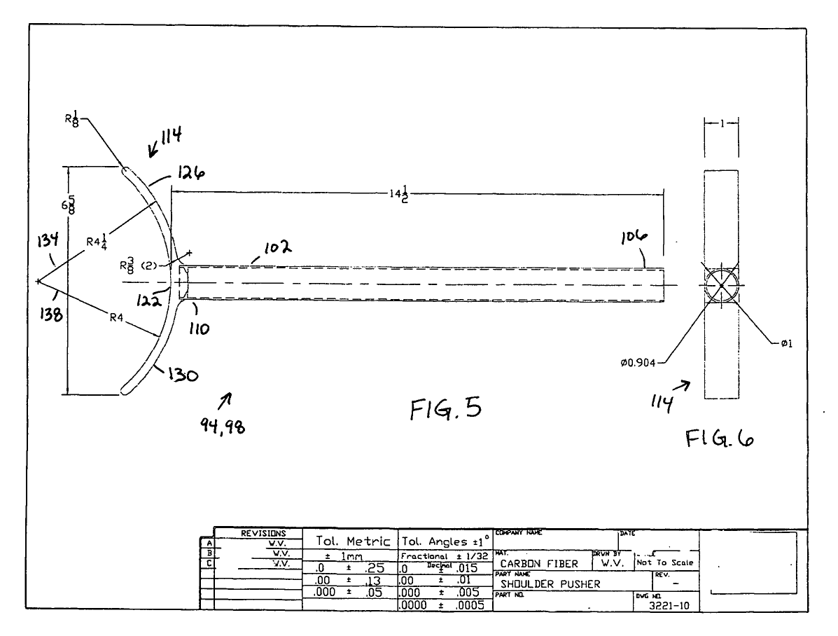

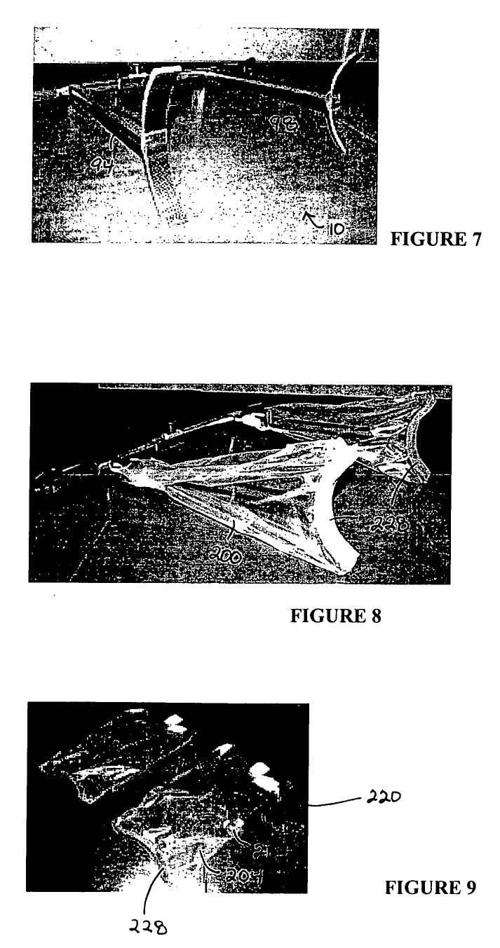

FIGS. 4-7 further illustrate the first arm 94 and the second arm 98. The first arm 94 includes an elongated rod 102 having a first end 106 and a second end 110. The elongated rod 102 includes a length generally defined by the distance between a person's head and shoulder. The first end 106 is adapted to be received within the first coupling 86 and be securely retained within the coupling with a friction fit. The elongated rod 102 has a substantially circular cross-section, but other shapes for the cross-section are also possible.

The first arm 94 also includes a base 114 coupled to the second end 110 of the rod 102. The base 114 is generally C-shaped and defines a recess 118 adapted to receive the shoulder of the patient. The base 114 includes a central portion 122 and a first extension 126 extending from the central portion 122 and a second extension 130 extending from the central portion 122. The first extension 126 defines a radius of curvature of a first length 134. The second extension 130 defines a radius of curvature of a second length 138. In one construction, the first length 134 and the second length 138 are substantially the same. In another construction, the first length 134 and the second length 138 are different. For example, the first length 134 can be about 4.25 inches and the second length 138 can be about 4.0 inches.

As illustrated in FIG. 2, the first arm 94 and the second arm 98 are spaced from one another based on a distance between the patient's left shoulder and right shoulder. As discussed above, the elongated rod 14 is adjusted to accommodate the width of the patient's shoulders. The area between the first arm 94 and the second arm 98 define a recess 142 adapted to receive the patient's head.

In operation, the device 10 is adjusted to accommodate the width of the patient's shoulders and is then applied to the patient's shoulders. When the patient is in a substantially horizontal position, the elongated rod 14 applies bilateral pressure to the patient's shoulders. In addition, the first arm and the second arm apply downward (i.e., toward the table the patient is lying on) pressure when pressure is applied to the elongated rod 14. When the downward pressure is applied to the elongated rod 14, the patient's shoulders are also pushed down and out of the way of the X-ray field. The device 10 allows for the acquisition of X-rays having C1 and C2, and C7 and C8, visible in a single X-ray.

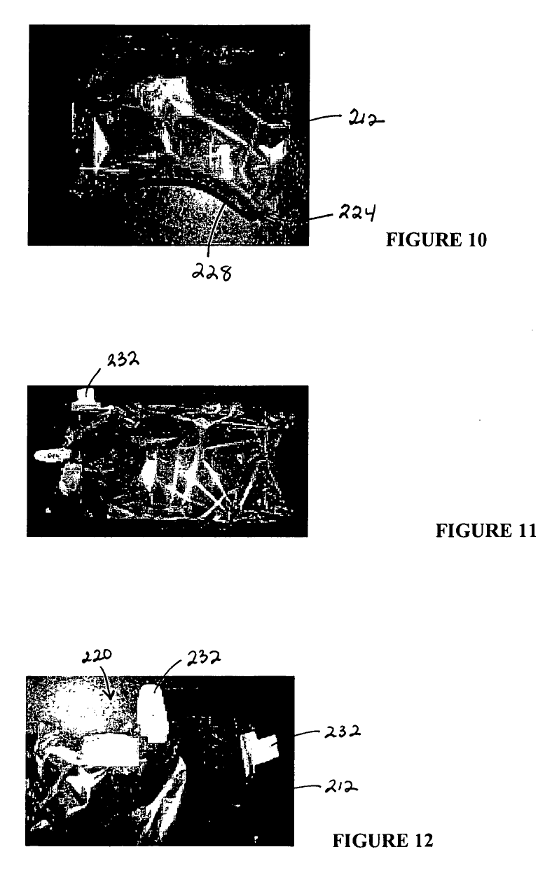

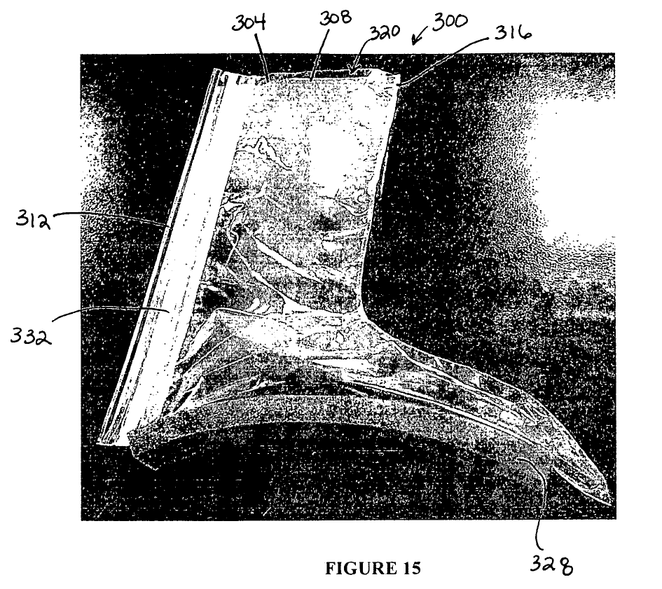

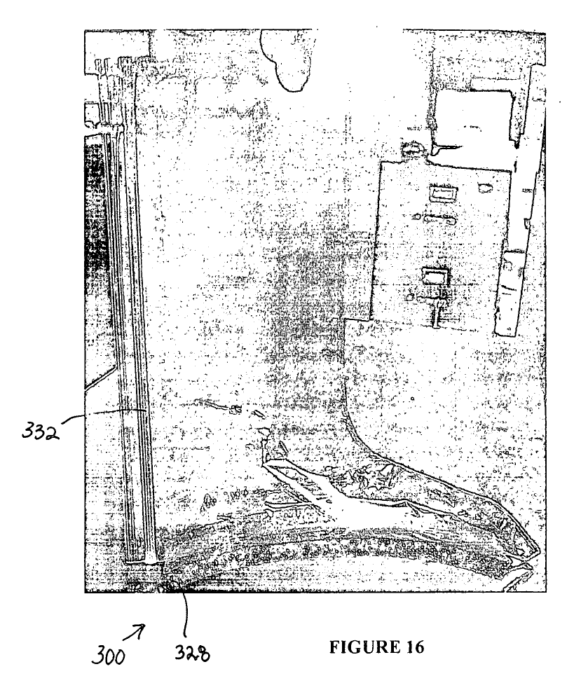

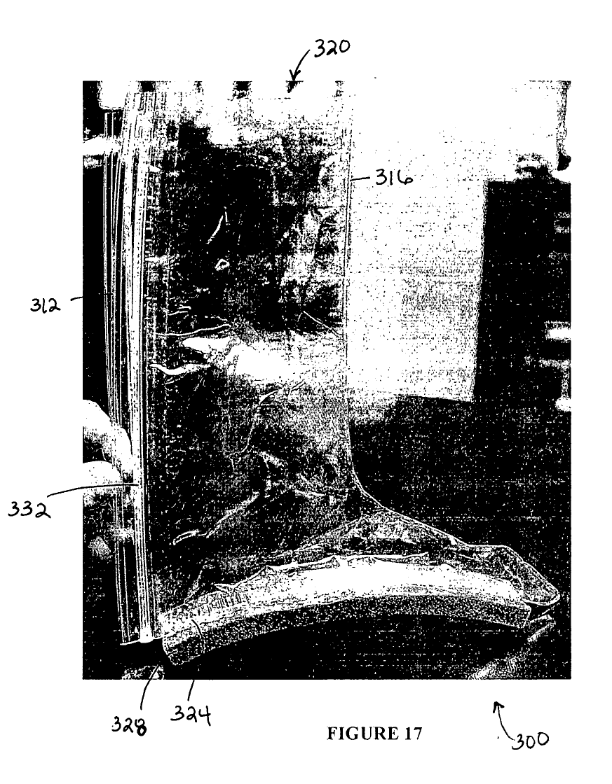

FIG. 8 illustrates a disposable sleeve 200 applied to the device 10 according to one embodiment of the present invention. The disposable sleeve 200 can be loose fitting (as illustrated in FIGS. 8-14) or form-fitted (as illustrated in FIGS. 15-17) to the specific portion of the device 10 upon which it is applied. The disposable sleeve 200 includes a first flexible side wall 204 and a second flexible side wall 208. The first flexible side wall 204 and the second flexible side wall 208 are joined at a first edge 212 and a second edge 216. The first flexible side wall 204 and the second flexible side wall 208 are comprised of a medical grade plastic or other suitable material. The material utilized for the side walls 204, 208 can be transparent and radiolucent. The disposable sleeve 200 includes an opening 220 at an upper end thereof. The opening 220 is adapted to receive one of the first arm 94 and the second arm 98 of the device 10.

With reference to FIGS. 9-11, the disposable sleeve 200 includes a bottom surface 224 opposite the opening 220 and extending between the first edge 212 and the second edge 216. The disposable sleeve 200 also includes a pad 228 connected to the bottom surface 224. The pad 228 includes a foam layer and can include more than one layer of the same or different materials. The pad 228 is compressible and does not adversely affect the imaging field because the foam layer includes a plurality of air chambers that do not appear in X-ray images. In one construction, the pad 228 can have a thickness in the range of about 0.25″ to about 1.5″. In other constructions, the pad 228 can have a thickness in the range of about 0.75″ to about 2.0 inches. Under compression (i.e., the device 10 is applied to a patient), the pad 228 has a thickness in the range of about 0.10″ to about 0.40″, and more preferably about 0.25″.

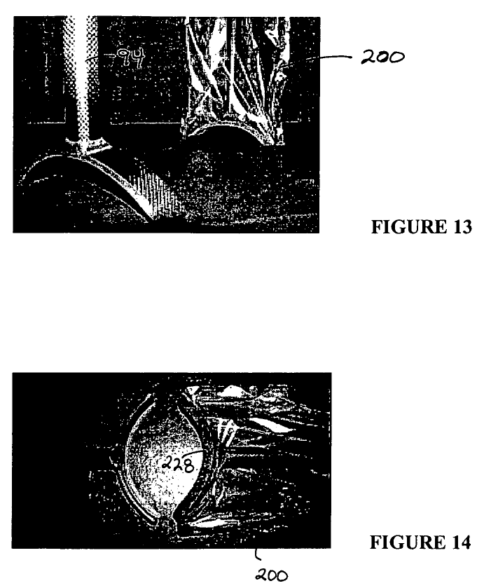

The pad 228 includes a radius of curvature substantially similar to the radius of curvature of the first extension 126 and the second extension 130 of the arms 94, 98 of the device 10. In this configuration, the disposable sleeve 200 is ready-to-use. The pad 228 retains its curved shape in the unused configuration such that the disposable sleeve 200 can be easily applied to the device 10 as illustrated in FIGS. 13-14.

The disposable sleeve 200 includes a plurality of tabs 232 connected adjacent to the first edge 212, the second edge 216, and the opening 220. The tabs 232 extend outward from the sleeve 200 and include a peel-off label to expose a layer of adhesive. The user applies the disposable sleeve 200 to the device 10 and secures the sleeve 200 to the device 10 with the adhesive on the tabs 232 as illustrated in FIG. 8.

FIGS. 15-17 illustrate a form-fitted disposable sleeve 300 for a modified device 10. The disposable sleeve 300 includes a first flexible side wall 304 and a second flexible side wall 308. The first flexible side wall 304 and the second flexible side wall 308 are joined at a first edge 312 and a second edge 316. The first flexible side wall 304 and the second flexible side wall 308 are comprised of a medical grade plastic or other suitable material. The material utilized for the side walls 304, 308 can be transparent and radiolucent. The disposable sleeve 300 includes an opening 320 at an upper end thereof. The opening 320 is adapted to receive one of the first arm 94 and the second arm 98 of the device 10.

The disposable sleeve 300 includes a bottom surface 324 opposite the opening 320 and extending between the first edge 312 and the second edge 316. The disposable sleeve 300 also includes a pad 328 connected to the bottom surface 324. The pad 328 includes a foam layer and can include more than one layer of the same or different materials. The pad 328 is compressible and does not adversely affect the imaging field because the foam layer includes a plurality of air chambers that do not appear in X-ray images. In one construction, the pad 328 can have a thickness in the range of about 0.25″ to about 1.5″. In other constructions, the pad 328 can have a thickness in the range of about 0.75″ to about 2.0 inches. Under compression (i.e., the device 10 is applied to a patient), the pad 328 has a thickness in the range of about 0.10″ to about 0.40″, and more preferably about 0.25″.

The pad 328 includes a radius of curvature substantially similar to the radius of curvature of the first extension 126 and the second extension 130 of the arms 94, 98 of the device 10. In this configuration, the disposable sleeve 300 is ready-to-use. The pad 328 retains its curved shape in the unused configuration such that the disposable sleeve 300 can be easily applied to the device 10.

The disposable sleeve 300 includes a recloseable seal 332 oriented along the first edge 312. The recloseable seal 332 can be used for ease of applying the disposable sleeve 300 to the device 10.

The embodiments described above and illustrated in the figures are presented by way of example only and are not intended as a limitation upon the concepts and principles of the present invention. As such, it will be appreciated by one having ordinary skill in the art that various changes in the elements and their configuration and arrangement are possible without departing from the spirit and scope of the present invention.

What is claimed is:

1. A disposable sleeve adapted for use with a shoulder position device, the disposable sleeve comprising:
a first flexible side wall;
a second flexible side wall connected to the first flexible side wall at a first edge and a second edge;
an opening at a first end of the sleeve and between the first flexible side wall and the second flexible side wall, the opening adapted to receive the shoulder position device; and
a pad secured to a second end of the sleeve, the pad defining a radius of curvature substantially similar to a radius of curvature of a portion of the shoulder position device.

2. The disposable sleeve of claim 1 wherein the pad includes a layer of foam.

3. The disposable sleeve of claim 2 wherein the pad is adapted to compress to a thickness in a range of about 0.10 inch to about 0.40 inch.

4. The disposable sleeve of clam 1 wherein the first flexible side wall and the second flexible side wall are comprised of medical grade plastic.

5. The disposable sleeve of claim 1 further comprising a tab secured to one of the first flexible side wall and the second flexible side wall, the tab including a peel-off label to expose adhesive.

6. The disposable sleeve of claim 5 wherein at least one of the tabs is positioned adjacent the opening of the sleeve.

7. The disposable sleeve of claim 5 wherein at least one of the tabs is positioned adjacent one of the first edge and the second edge.

8. The disposable sleeve of claim 1 further comprising a recloseable seal along one of the first edge and the second edge.

9. A disposable sleeve adapted for use with a shoulder position device, the disposable sleeve comprising:
a first flexible side wall;
a second flexible side wall connected to the first flexible side wall at a first edge and a second edge;
an opening at a first end of the sleeve, the opening adapted to receive the shoulder position device;
a pad secured to a second end of the sleeve, the pad defining a radius of curvature; and
a plurality of tabs secured to the sleeve, the tabs having a layer of adhesive adapted to be secured to a portion of the shoulder position device.

10. The disposable sleeve of claim 9 wherein the radius of curvature of the pad is substantially similar to a radius of curvature of a portion of the medical device.

11. The disposable sleeve of claim 9 wherein the pad includes a layer of foam.

12. The disposable sleeve of claim 11 wherein the pad is adapted to compress to a thickness in a range of about 0.10 inch to about 0.40 inch.

13. The disposable sleeve of clam 9 wherein the first flexible side wall and the second flexible side wall are comprised of medical grade plastic.

14. The disposable sleeve of claim 9 wherein the tabs are secured to one of the first flexible side wall and the second flexible side wall, the tab including a peel-off label to expose the layer of adhesive.

15. The disposable sleeve of claim 14 wherein at least one of the tabs is positioned adjacent the opening of the sleeve.

16. The disposable sleeve of claim 14 wherein at least one of the tabs is positioned adjacent one of the first edge and the second edge.
